(19) United States
(12) Patent Application Publication
(10) Pub. No.: US 2021/0215710 A1
(43) Pub. Date: Jul. 15, 2021

(54) METHODS FOR USE OF CDX2 AS A BIOMARKER TO GUIDE USE OF AUTOPHAGY INHIBITORS IN OVARIAN CANCER

(71) Applicants: UNIVERSITY HEALTH NETWORK, TORONTO (CA); SINAI HEALTH SYSTEM, TORONTO (CA)

(72) Inventors: IGOR JURISICA, TORONTO (CA); ANDREA JURISICOVA, TORONTO (CA)

(21) Appl. No.: 17/133,257

(22) Filed: Dec. 23, 2020

Related U.S. Application Data
(60) Provisional application No. 62/952,724, filed on Dec. 23, 2019.

(51) Int. Cl. G01N 33/68; A61K 31/4706; C12Q 1/6886; A61K 31/52

(52) U.S. Cl. CPC G01N 33/6872; A61K 31/4706; G01N 2333/4748; A61K 31/52; C12Q 1/6886

(57) ABSTRACT

Provided are methods of selecting a patient and/or treating a patient afflicted with high-grade serous epithelial that comprises upregulation of CDX2 and for autophagy inhibitor treatment. For example, the method of selecting a patient afflicted with a cancer likely to benefit from an autophagy inhibitor treatment, includes obtaining a biological sample; testing the biological sample for upregulated CDX2; and selecting the patient having a upregulated CDX2, as likely to benefit from and/or for treatment with the autophagy inhibitor treatment. Also provided are methods involving mutational status of p53.

This application claims the benefit of priority of U.S. Patent Application Ser. No. 62/952,724, filed on Dec. 23, 2019, incorporated herein by reference.

### FIELD

The present disclosure relates to methods and compositions for identifying patients who would benefit from treatment and/or treating patients with an autophagy inhibitor and more particularly to identifying patients having upregulated caudal-related homeobox transcription factor 2 (CDX2) levels, for treatment with an autophagy inhibitor, for example, chloroquine.

### BACKGROUND

Epithelial Ovarian Cancer (EOC) remains the most lethal gynecologic malignancy, accounting for 2,600 new cases and more than 1,750 deaths a year in Canada alone. If cancer is detected early, more than 80% of women will respond to therapy and survive. Unfortunately, early diagnosis is difficult due to the physically inaccessible location of the ovaries, the vagueness of symptoms during early disease (such as abdominal swelling, discomfort, pain), and the limited understanding of ovarian oncogenesis^{2, 3}. As a result, approximately 70% of cases are identified at advanced stages, when disease has spread from the ovaries to the peritoneal cavity. Clinical progress remains hampered due to a lack of effective biomarkers for detecting early-stage disease, reliable prognostic markers for identifying high risk patients, and markers for predicting clinical response to treatment.

CDX2 is a transcription factor, a protein that binds to DNA at a specific site to regulate the expression of particular gene(s). CDX2 regulates the expression of homeobox genes, that govern various differentiation processes in epithelial cells and affect processes such as cell proliferation and migration.

In low-grade ovarian cancer such as most mucinous and endometrial ovarian cancers, CDX2 overexpression is associated with better outcomes. CDX2 is also known to correlate with favorable outcomes in other epithelial cell cancers.

CDX2 expression has been studied as a biomarker, and was found to differentiate metastatic from primary ovarian cancer cells [Kim, J. Korean Acad. Sci., 2005, 20:643-8].

In colorectal cancer reduced CDX2 expression is associated with more advanced tumor stage and metastasis [Graule et al. Clinical Epigenetics (2018) 10:120]. In gastrointestinal cancer, the prognostic role of CDX2 is unclear [Masood et al., Acta Gastroenterol Belg. 2016 April-June; 79(2):197-200].

The role of chloroquine and its analogs as autophagy inhibitors in cancers is reviewed in [Kimura et al., Cancer Research; 73(1); 3-7, 2012], citing the elevated risk of acute kidney injury as a barrier to the broad use of these therapies in cancer. A broader review of autophagy inhibition is provided in [Al-Bari, J Antimicrobial Chemotherapy, 2015; 70: 1608-1621, 2015]. Low concentration of chloroquine and cisplatin have been tested as a treatment in ovarian cancer [Zhu et al., Am J Transl Res 2017; 9(9):4046-4058, 2017] and autophagy inhibition reduces chemoresistance in human ovarian cancer stem cells [Pagotto et al., Cell Death and Disease (2017) 8, e2943]. Re-purposing chloroquine for glioblastoma and the confounding variables is provided in [Weyerhauser et al., FRONTIERS IN ONCOLOGY VO. 8, NO. 335, August 2018]. An overview of repurposing mefloquine for cancer treatment is provided in [Mereddy and Ronayne, Transl Med (Sunnyvale), 2018, 8:1]. Mefloquine induces cell death in prostate cancer cells in [Yan et al., ONCOLOGY LETTERS VOL. 5, pp. 1567-1571, 2013]. Lucanthone has been reported to inhibit autophagy [Carew et al., THE JOURNAL OF BIOLOGICAL CHEMISTRY VOL. 286, NO. 8, pp. 6602-6613, February 25, 2011] a [Carew and Nawrocki, AUTOPHAGY, 2017, VOL. 13, NO. 4, 765-766]. Autophagy inhibitors to treat cancer are described in EP 2 544 673 B1.

Personalized treatments for treating ovarian cancer are desirable.

### SUMMARY

CDX2 overexpression, in ovarian cancer cells is identified herein as a biomarker for the use of autophagy inhibition to treat patients with high-grade serous ovarian cancer. As described in the Examples, CDX2 overexpression was found to activate autophagy in cells, suggesting that CDX2+ overexpression is a biomarker that can be used to stratify patients that could benefit from autophagy inhibition. Disclosed herein are methods for personalizing cancer treatment of patients having high-grade serous epithelial ovarian cancer with an upregulation of CDX2, for example due to amplification of CDX2.

According to an aspect of the invention, there is provided a method of selecting a patient afflicted with high-grade serous epithelial ovarian cancer likely to benefit from an autophagy inhibitor treatment, the method comprising:

obtaining a biological sample;
testing the biological sample for upregulated CDX2; and
selecting the patient having upregulated CDX2 as likely to benefit from and/or for treatment with the autophagy inhibitor treatment.

Also provided in another aspect, is a method of treating a patient afflicted with high-grade serous epithelial ovarian cancer having upregulated CDX2, the method comprising administering to said patient an autophagy inhibitor treatment.

A further aspect provides a method of treating a patient afflicted with high-grade serous epithelial ovarian cancer, the method comprising:
obtaining a biological sample;
testing the biological sample for upregulated CDX2; and
treating the patient having upregulated CDX2 with an autophagy inhibitor treatment.

It is also demonstrated herein that upregulated CDX2 and loss of function of p53 protein, optionally one or more deleterious mutations in p53, can be an indicator of poorer prognosis of the patients with serous ovarian cancer. Some embodiments further comprise testing the sample for loss of function of p53, optionally testing for one or more deleterious mutations in p53.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1A-C depicts serous ovarian cancer patients with high CDX2 in the TOGA dataset who have significantly poorer disease-free survival (P<0.03). FIG. 1A illustrates mean survival rate for patients with high CDX2 expression is 19% compare to the mean survival of the patients with low CDX2 expression. FIG. 1B illustrates further survival analysis in cluster of patients with known p53 mutation (HR=1.64; P=0.042). FIG. 10 shows that patients with high CDX2 expression but non-mutated P53 have better survival (HR=0.59;0020P=0.078).

FIG. 2 is a network analysis of CDX2-p53 axis in serous ovarian cancer. Transcriptional (gene expression) profiles of 839 samples of ovarian cancer were analyzed to identify genes that are differentially expressed in ovarian cancer. This data was overlaid on proprietary maps linking transcription factors (here CDX2) to the specific genes whose transcription they regulate, and of similar maps linking genes through common pathways and protein-protein interaction networks (known associations that execute biological functions). Known interactions from the literature. and novel interactions are identified from the inventor's proprietary biological network maps.

FIG. 3A-C are images that depict Live and dead ovarian cancer cells in hanging drop experiments to confirm autophagy activation in CDX2(+) cells. FIG. 3A depicts a CDX2(−) parental control. FIGS. 3B and 3C depict CDX2(+) derived cell lines. The mouse ovarian cancer cell line MOSEC5 that is CDX2(−) was genetically modified to create CDX2(+) derived cell lines. Cells were placed in a “hanging drop” assay under starvation conditions. Living cells are shown in green and dead cells are red, demonstrating that CDX2(+) cells survive longer. Metabolite analysis confirmed the activation of autophagy in CDX2(+) cells. FIG. 3D illustrates human cell lines OVCA 429 with and without CDX2.

FIG. 4A depicts a line graph illustrating the survival of mice with CDX2(+) or CDX2(−) ovarian cells implanted. FIG. 4B depicts bar graphs illustrating volume of ascites/mouse in mice with CDX2(+) or CDX2(−) ovarian cells implanted. FIG. 4C depicts images of CDX2(−) and CDX2(+) ovarian tumors and their size. FIG. 4D depicts images illustrating the invasiveness of CDX2(−) and CDX2(+) tumors. Surgical implantation of CDX2(−) and CDX2(+) ovarian cancer cells in mice show that CDX2(+) cells generate larger tumors that aggressively spread to ascites and surrounding tissue, decreasing survival. CDX2(+) tumors reduce survival, lead to increased volume of ascites, are larger, and are more invasive of surrounding tissue.

FIG. 5 shows a protein interaction network using data from a transcriptional profile screen to identify chloroquine as an autophagy inhibitor. The only common “linkers” between CDX2 and up- down-regulated genes include TP53 and TOP1. Screening of existing drugs for their activity to reverse autophagy found three candidates, including chloroquine (shown here).

FIG. 6A depicts bar graphs that illustrate the long-term effect of nutritional deprivation on cellular survival in CDX2(−) and CDX2(+) cell colonies in vitro and the effect of treatment with autophagy inhibitors. This illustrates the effect of autophagy inhibition on CDX2(+) cancer cell survival. Survival under starvation is far superior for the CDX2(+) derived cell lines D4 and B2 than the CDX2(−) cell line Gal. However, upon administration of autophagy inhibitors, the survival of CDX2(+) cells is dramatically reduced. CDX2(+) show increased survival after long-term stress, but chloroquine (CQ) and the autophagy inhibitor 3-methyladenine (3MA) reverse this survival. P<0.001.

FIG. 6B depicts the administration of chloroquine (10 mg/kg 3× per week) was initiated 30 days after delivery of tumor cells, which resembles well established cancer. For all experiments aged females (9-10 month) were used, as these represent targeted patient population (e.g., peri-menopausal women). Mice were sacrificed when ascites formed. In the graph, the first line/bar is the vehicle and the second line/bar is chloroquine.

FIG. 7 depicts bar graphs that illustrate the results of an OVCA429 soft agar assay. Chloroquine (CQ) decreased advantage of CDX2-driven anchorage-independent growth (4 weeks) in OVCA429. This confirms the effect in human high grade serous ovarian cancer cells. Similar to mouse model, Cdx2 over expression in human ovarian cancer cells increased cell survival under nutritional deprivation and treatment with chloroquine abrogated this selective growth advantage. CDX2(−) at left. 48 h IC50 CQ in adherent culture: GAL is 11.46 uM and CDX2 is 9.3 uM. P<0.005.

FIG. 8 depicts a bar graph that illustrates acute cell survival is facilitated by CDX2. Increased proportion of CDX-2 positive cells activate autophagy even under optimal growth conditions. This is further increased when glucose and serum are removed from culture conditions (48 h).

FIG. 9 is an image depicting long-term survival persisted even after 7 days of nutritional deprivation. This was confirmed upon re-feeding remaining cells in normal growth media.

FIG. 10 is an image depicting CDX2 activates autophagy even in abundant nutritional conditions with further increase throughout 17-48 h starvation period. This is evident by increased lipidation of LC3 (LC3/II) and formation of LC3 foci in cells.

FIG. 11 is an image depicting LAMP1, integral component of lysosomal membranes and a member of CLEAR gene signature, has increased glycosylation profile, evidenced by higher molecular weight.

FIG. 12 is a graph depicting CDX2 is over-expressed in 10.8% of serous EOC in average (top bar; range 5.5%-18.2%); public TOGA and GEO.

FIG. 13A demonstrates lower survival of patients with ovarian serous cancer with high level of Cdx2 expression and autophagy genes Atg5, Atg12 and Becn1) and lysosomal/autophagy genes Top1, Top2A, Apex1).

FIG. 13B demonstrates lower disease free-survival of patients with ovarian serous cancer when lysosome related genes Ubb, Ubc, Pin1 and Cdx2 are over-expressed. The Cancer Genome Atlas (TCGA) Ovarian Serous Cystandenocarincoma Gene Expression Data; accessed via MSKCC cBioPortal for Cancer Genomics (http://www.cbioportal.org/public-portal/).

FIG. 13C demonstrates increased disease-free survival of patients with ovarian serous cancer with over-expressed CbX4, Tnf, Ubc, Src, Grb2 when treated by chloroquine.

### DETAILED DESCRIPTION OF THE DISCLOSURE

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, e.g. Green and Sambrook, 2012).

### Definitions

As used herein “autophagy” refers to the cell mechanism where cellular components are degraded and recycled, including for example, stress-induced autophagy, and can for example, be evidenced by LC3 lipidation and/or decreased levels of p62 (a substrate for autophagy)

As used herein an “autophagy inhibitor” means any molecule including a compound (e.g. small molecule drug), that interferes with autophagy formation and completion, including formation of the autophagosome, its docking and fusion to the lysosome, and/or the function of the autolysosome, including for example ROC-325, mefloquine, lucanthone, 3-methyladenine and their analogs. Also included are autophagy inhibitors described in EP 2544673B1 incorporated herein by reference. As used herein, “autophagy inhibitor treatment” includes administering one or more autophagy inhibitor(s) to a patient.

As used herein “ascites” refer to an accumulation of fluid in the abdominal cavity, associated with epithelial ovarian cancer, which comprise cellular and acellular components. Examples of the cellular components include tumor cells, optionally single cells or spheroid cells, and stroma cells, including fibroblasts, inflammatory cells, mesothelial cells, endothelial cells, adipocytes, and/or adipose tissue derived stromal cells. Examples of acellular components include cytokines, proteins, metabolites, and/or exosomes. For example, ascites cellular fraction may comprise any of the cellular components of ascites.

As used herein, “circulating tumor cells” refer to tumor cells that are present in non-solid biological tissue, for example, blood.

As used herein, “circulating tumor DNA” refers to DNA or fragments thereof from tumor cells that are circulating in non-solid biological tissue, for example blood.

As used herein, “liquid biopsy” refers to the sampling and analysis of non-solid biological tissue, for example blood. A liquid biopsy may, for example, be performed to isolate cancer cells from a tumor that are circulating in the blood, or fragments of DNA from tumor cells that are in the blood.

As used herein, “CDX2” refers to Caudal-related homeobox transcription factor 2 (e.g. Gene ID: 1045), including all natural and mutant forms thereof, including any splice variants.

As used herein “chloroquine” means a compound having the following structure:

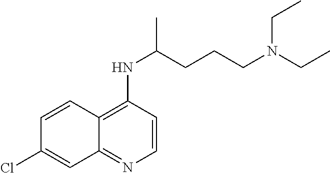 [SMILES: CCN(CC)CCCC(C)Nc1ccnc2cc(Cl)ccc12]

and pharmaceutically acceptable salts thereof.

As used herein “analog” includes structurally related molecules that have a similar or better potency and/or other biological property as the base compound, which in the context of the present autophagy inhibitors includes analogs that interfere with fusion of the autophagosome and the lysosome and/or inhibits autophagosome formation via the inhibitor of class III PI3K and/or lysosomal function. For example, analogs of chloroquine include, hydroxychloroquine, quinacrine, and 8-hydroxyquinoline. Analogs of chloroquine and mefloquine can be found for example in WO2010144434, US20050154010 WO2019200284, WO2010144102, incorporated herein by reference.

As used herein “quinacrine” refers to a compound having the following structure:

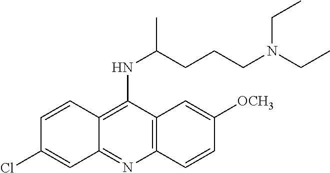 [SMILES: CCN(CC)CCCC(C)Nc1c2ccc(Cl)cc2nc2ccc(OC)cc12]

and pharmaceutically acceptable salts thereof.

As used herein “hydroxychloroquine” refers to a compound having the following structure:

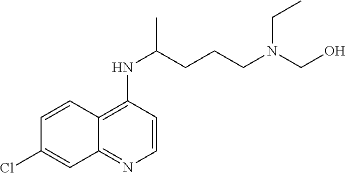 [SMILES: CCN(CO)CCCC(C)Nc1ccnc2cc(Cl)ccc12]

and pharmaceutically acceptable salts thereof.

As used herein “hydroxychloroquine” refers to a compound having the following structure:

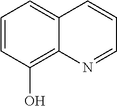 [SMILES: Oc1cccc2cccnc12]

and pharmaceutically acceptable salts thereof.

As used herein “ROC-325” refers to a compound having the following structure:

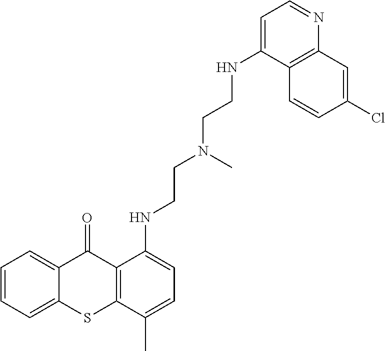 [SMILES: Cc1ccc(NCCN(C)CCNc2ccnc3cc(Cl)ccc23)c2c(=O)c3ccccc3sc12]

and pharmaceutically acceptable salts thereof.

As used herein “mefloquine” refers to a compound having the following structure:

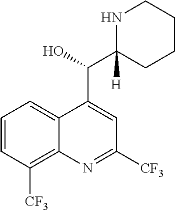 [SMILES: O[C@@H](c1cc(C(F)(F)F)nc2c(C(F)(F)F)cccc12)[C@H]1CCCCN1]

and pharmaceutically acceptable salts thereof.

As used herein “lucanthone” refers to a compound having the following structure:

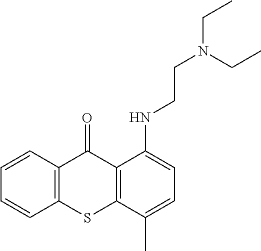 [SMILES: CCN(CC)CCNc1ccc(C)c2sc3ccccc3c(=O)c12]

and pharmaceutically acceptable salts thereof.

As used herein “3-methyladenine” means a molecule that inhibits autophagy and has the following chemical structure:

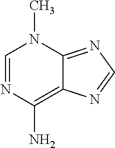

and pharmaceutically acceptable salts thereof.

As used herein “high-grade serous epithelial ovarian cancer” means a type of epithelial ovarian cancer characterized by high-grade tumors, which are aggressive, grow rapidly, and often spread beyond the ovary, and result in shorter disease-free survival period compared to for example low-grade disease such as most mucinous and endometrial ovarian cancers.

As used herein “a biological sample” means any sample from a subject such as a human and comprises, depending on the assay, cancer cells or a fraction thereof, for example tumor cells, including for example a tumor tissue sample such as a biopsy, tissue slice, or cancer cells, such as blood cancers circulatory tumor cells, which can be obtained by liquid biopsy. Fractions thereof include for example, nucleic acid fractions, (e.g. genomic DNA or mRNA) for testing for genetic alterations or protein fractions.

As used herein, the term “likely to benefit” refers to an increased probability or greater likelihood that a particular treatment will have positive therapeutic effect on a patient. For example, a patient having a tumor with a deleterious mutation in the p53 and an upregulation of CDX2 is more likely to respond to an autophagy inhibitor than someone without the mutation and upregulation and is considered likely to benefit compared to a patient without the mutation and CDX2 upregulation.

As used herein, the term “upregulation” or upregulated refers to an increased quantity and/or activity of a cellular component, for example, DNA, RNA or protein, (such as in the case of CDX2 is being overexpressed), that is for example at least 50% greater than a corresponding reference cell where the cellular component quantity or activity is not upregulated and/or which is detectable when compared to a corresponding reference cell where such quantity or activity is not detectable.

The term “biomarker”, as used herewith, refers to a measurable substance or alteration in a substance for example a deleterious genetic alteration such as a deleterious mutation, or amplification, which is characteristic for a specific situation, for example, associated with increased beneficial response to a treatment of inhibitor.

As used herein, the terms “patient” or “subject” may be used interchangeably herein, and refer to a mammalian subject, and preferably to a human.

The terms “treat” or “treating”, as used herein, unless otherwise indicated, mean reversing, alleviating, inhibiting the progression of, or preventing the disorder or condition to which such term applies, or one or more symptoms of such disorder or condition. The term “treatment”, as used herein, unless otherwise indicated, refers to the act of treating, as defined immediately above.

The term “effective amount” as used herein is an amount of an inhibitor that is sufficient to reduce cell growth or proliferation of cells, and/or which alleviates at least one symptom as found for the disease to be treated. Alleviating is meant to include, e.g., treating, reducing the symptoms of, or curing the disease or condition.

“Tumor”, as used herein, refers to all neoplastic masses of tissue, and all pre-cancerous and cancerous tissue growths.

The terms “cancer” and “cancerous” refer to any malignant and/or invasive proliferation, growth or tumor caused by abnormal cell growth. As used herein “cancer” includes solid tumors named for the type of cells that form them, cancer of blood, bone marrow, or the lymphatic system. The term cancer includes, but is not limited to, a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one.

As used herein, the term “cancer cells” refer to cells of a cancer, e.g. cells that acquire a characteristic set of functional capabilities during their development, including the ability to evade apoptosis, self-sufficiency in growth signals, insensitivity to anti-growth signals, tissue invasion/metastasis, significant growth potential, and/or sustained angiogenesis.

The term “afflicted” includes subjects suffering from the disease and/or diagnosed with the disease.

The term “select” with respect to selecting a patient, for example for treatment, to be engaged for testing for personalized medicine, or participate in a clinical trial refers to a physical selection, for example, a medical professional or allied health worker providing an indication of the selection, for example in written form, computer communication, or verbal communication.

The term “deleterious mutations” as used herein refers to a change in the sequence of a gene that negatively effects the function of the encoded molecule and which can be reflected for example in the activity or level of transcript/protein. As used herein, such deleterious mutations include mutations such as deletion mutations, deep deletions, point mutations, insertion imitations, or missense mutations, including those that cause protein truncation, that result in loss of function of the gene product (such as in the case of p53).

As used herein with reference to a gene abnormality, the terms “amplification” refers to the presence of a higher than normal number of copies of a genomic nucleic acid sequence and includes multiple copy gain (such as in the case of CDX2).

As used herein, “surgical sample” refers to a biological sample that is obtained surgically, for example a biopsy.

The term “biopsy” as used in the description of the invention includes all types of biopsies known to those skilled in the art. Thus, the term “biopsy”, as used in the context of the present invention, may include, for example, samples obtained by resection of tumors, tissue samples obtained by endoscopic methods, or organ biopsies obtained using forceps or a needle, or liquid biopsy such as circulating tumor cells or circulating tumor DNA.

As used herein “wild-type” in reference to a nucleic refers to a naturally occurring sequence of a nucleic add at a genetic locus, for example in the genome of an organism, or a sequence transcribed or translated from such a nucleic acid.

The term “assay” as used herein refers to a procedure used for the quantitative or qualitative analysis of an analyte.

As used herein, the term “standard polypeptide assay” refers to assays used to detect or measure a level of a polypeptide. Many such assays are known to those skilled in the art, including, for example, Western blots, immunoblots, enzyme-linked immunosorbent assays (ELISAs), including competitive ELISAs, radioimmunoassay (RIA), surface plasmon resonance, fluorescence activated cell sorting (FACS), and flow cytometry.

As used herein, the term “point mutation” refers to the existence a nucleotide change (i.e. mutation) at a site relative to a wildtype sequence and/or the identity of the nucleotide present at the site of the mutation in the mutant copy of a genomic locus. The nucleotide can be in any chain of a double stranded DNA molecule.

As used herein, the term “truncation” refers to a shortening in the amino acid sequence of protein. A protein truncation may be the result of a truncation in the nucleic acid sequence encoding the protein, a substitution or other mutation that creates a premature stop codon without shortening the nucleic acid sequence, or from alternate splicing of RNA in which a substitution or other mutation that does not itself cause a truncation results in aberrant RNA processing.

The term “deletion” in the context of deletion mutants as used herein, refers to the removal or loss of one or more nucleotides from a nucleic acid sequence and includes for example deep deletions,

As used herein, the term “loss of function of p53” refers to a p53 protein that lacks or has decreased biological function, such as or specifically its ability to act as a tumor suppressing protein.

As used in this specification and the appended claims, the singular forms “a”, “an” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The definitions and embodiments described in particular sections are intended to be applicable to other embodiments herein described for which they are suitable as would be understood by a person skilled in the art. For example, in the following passages, different aspects are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Methods and Kits

Accordingly, an aspect includes a method of selecting a patient afflicted with high-grade serous epithelial ovarian cancer likely to benefit from an autophagy inhibitor treatment, the method comprising:
obtaining a biological sample;
testing the biological sample for upregulated CDX2; and
selecting the patient having a upregulated CDX2, as likely to benefit from and/or for treatment with the autophagy inhibitor treatment.

In an embodiment, the method comprises testing a patient afflicted with high-grade serous epithelial ovarian cancer also for loss of function of p53 protein, optionally testing for one or more deleterious mutations in the p53 protein, and selecting the patient having upregulated CDX2 and loss of function of the p53 protein for treatment with the autophagy inhibitor treatment.

A further aspect provides a method of treating a patient afflicted with high-grade serous epithelial ovarian cancer, the method comprising:
obtaining a biological sample;
testing the biological sample for upregulated CDX2; and
treating the patient having upregulated CDX2, with an autophagy inhibitor treatment.

Also provided in another aspect, is a method of treating a patient afflicted with high-grade serous epithelial ovarian cancer having upregulated CDX2, the method comprising administering to the patient an autophagy inhibitor treatment. In another aspect, the method comprises testing a patient afflicted with high-grade serous epithelial ovarian cancer also for a loss of function of p53 protein, optionally testing for one or more deleterious mutations in the p53 protein, and treating the patient with an upregulated CDX2 and a loss of function of the p53 protein with an autophagy inhibitor treatment. The autophagy inhibitor treatment is a treatment that includes an autophagy inhibitor and optionally one or more other agents, such as one or more chemotherapeutics. Chloroquine, mefloquine, lucanthone, ROC-325, and 3-methyladenine are autophagy inhibitors.

Accordingly, in another aspect, the autophagy inhibitor is an inhibitor that targets P13 kinase, autophagosome-lysosome fusion and/or autolysosome function. In another embodiment, the autophagy inhibitor is selected from ROC-325, 3-methyladenine, chloroquine, mefloquine, or lucanthone or their analogs or combinations thereof. In a further embodiment, the autophagy inhibitor is 3-methyladenine. In another embodiment, the autophagy inhibitor is chloroquine.

The biological sample can be any sample from the patient comprising cancer cell molecules that can be used for assessing upregulation of CDX2 and optionally mutational status of p53. For example, the biological sample can comprise cancer cell nucleic acids, optionally tumor genomic DNA or cancer cell transcripts, and can include a tumor sample, for example obtained during surgery, cellular fraction from ascites or a liquid biopsy comprising circulating tumor cells. The liquid biopsy can be a blood sample, or a fraction thereof, for example circulating tumor cell fraction, or nucleic acid fraction. The samples can be subjected to one or more steps to isolate nucleic acids. The biological sample can also comprise a cellular protein extract, for example in methods that test for CDX2 protein levels. In such case a tumor sample or in the case of blood cancers, a blood sample, can be taken and a cancer cellular protein fraction isolated.

Accordingly, in some embodiments, the biological sample comprises cancer cell nucleic acids. In other embodiments, the biological sample comprises cancer cell protein fraction.

In some embodiments, the testing comprises measuring the cellular levels of the CDX2 protein or mRNA. In an embodiment, the cellular levels of the CDX2 mRNA are detected by RT-PCR or qPCR methods. In a further embodiment, the cellular levels of the CDX2 protein are detected using a standard polypeptide assay, for example, an ELISA or Western Blot, or by immunohistochemistry of a tumor sample or immunocytochemistry of a cell sample.

CDX2 upregulation can be assayed by assessing for gene amplification of CDX2. For example, gene amplification can be detected using qPCR, RNAseq, and/or fluorescence in situ hybridization (FISH).

In some embodiments, the testing comprises assaying for one or more deleterious mutations in p53. For example, the testing can comprise sequencing a p53 transcript or part thereof to detect the mutational status of p53.

In another embodiment, testing further comprises comparing the p53 sequence to wild type p53 to identify the presence or absence of one or more deleterious mutations.

In another aspect, the method further comprises treating the patient with an effective amount of an autophagy inhibitor as described herein. The treatment can be combined with one or more suitable treatments such as a chemotherapeutic treatment.

Also provided are uses, for example use of an autophagy inhibitor treatment for treating a patient afflicted with a cancer having an upregulation of CDX2, for example from an amplification of CDX2.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

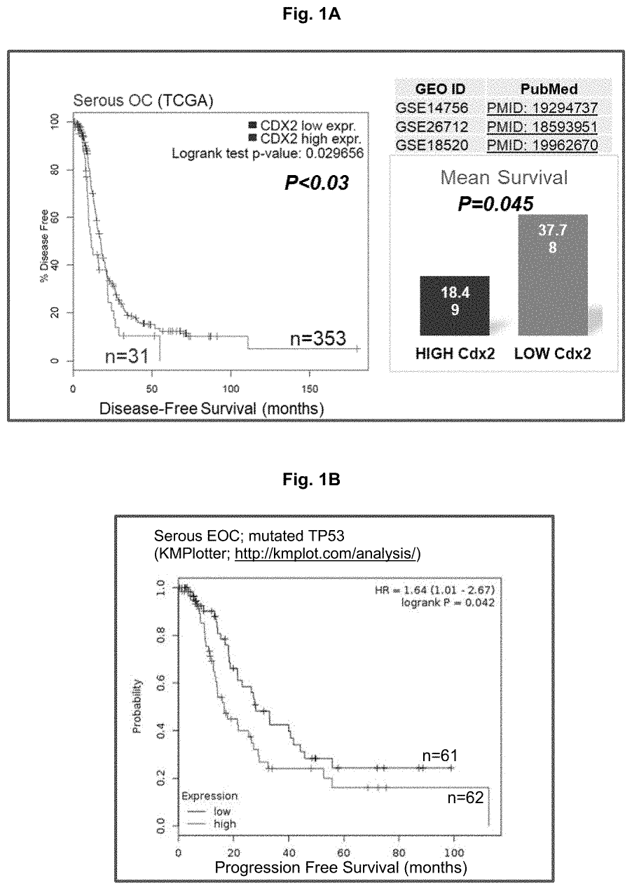

Analysis of data in The Cancer Genome Atlas suggested CDX2 overexpression in combination with mutation of the p53 tumor suppression gene signifies poor survival in women with high-grade serous ovarian cancer FIG. 1. CDX2 is far from being obvious as a biomarker, because in low-grade disease (most mucinous and endometrial ovarian cancer) CDX2 overexpression is associated with better outcomes. It is only in serous high-grade ovarian cancer that CDX2 overexpression makes outcomes poorer. In other epithelial cell cancers, CDX2 is also known to correlate with favorable outcomes; therefore, the unique role of CDX2 in high-grade serous ovarian cancer stems from a complex network-level effect, observed only when both CDX2 is overexpressed and p53 mutated.

Experimental analysis of tumor confirmed CDX2 is overexpressed in serous ovarian cancer patients who died of disease, relative to those who survived. With this confirmation, transcriptional profiles of 839 samples were obtained to measure differential gene expression and these data were mapped to proprietary maps of biological networks created by combining datamining of the literature with predictive algorithms to fill in gaps in the literature.

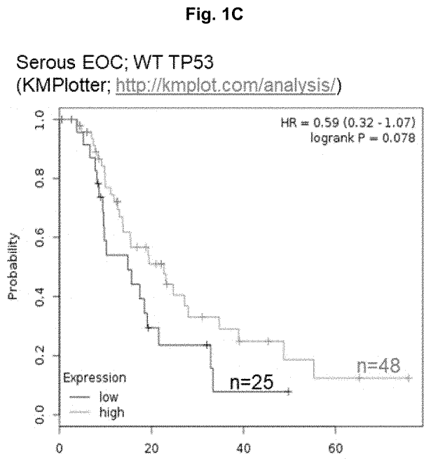

This network mapping identified particular genes linking CDX2 and p53 that are active in the subset of women with poor clinical outcomes (CDX2(+) and mutated p53) (FIG. 2). From this network analysis, specific genes (and their proteins) were identified that are implicated in disease involvement when CDX2 is expressed and p53 is mutated. Consulting gene ontology databases, it was found that these genes are associated with mTOR signaling and autophagy, giving rise to possible mechanisms underpinning the poor survival in patients with overexpression of CDX2 (CDX2(+)) and mutant p-53 (mut-p53). These genes were found to be associated with autophagy, a process by which cells can turn off parts of their machinery in order to save energy when faced with starvation conditions. This is of clinical relevance in ovarian cancer, as current standard of care is surgery followed by chemotherapy. If cancer cells activate autophagy, they are better suited to survive in ascites (fluid in the abdominal cavity) long enough to migrate to other locations and there metastasize.

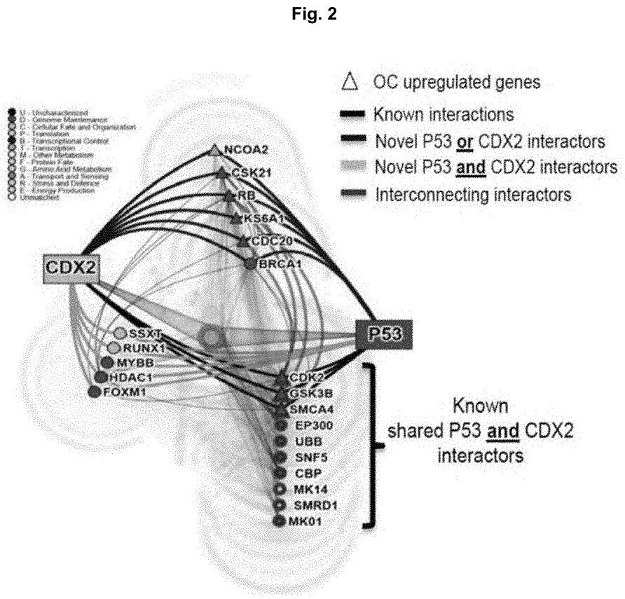

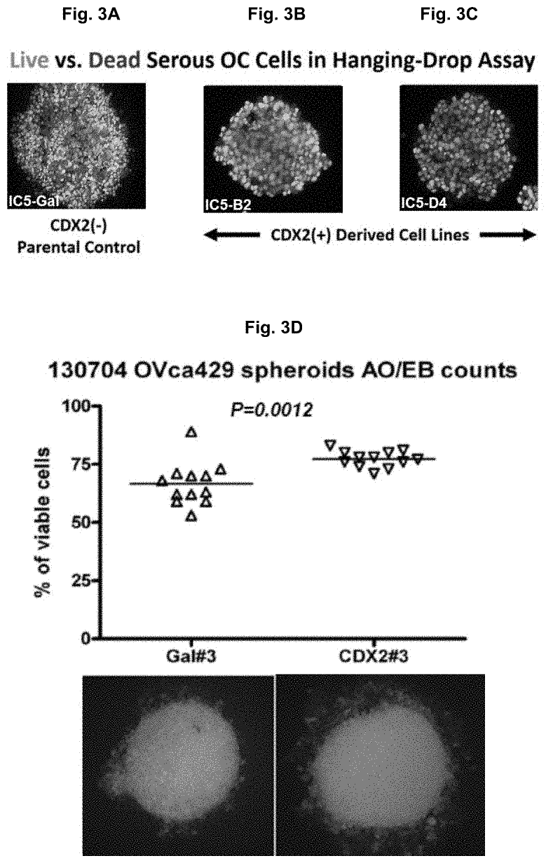

In vitro hanging drop assay experiments were conducted in which CDX2(+) ovarian cancer cells were found to activate autophagy and survive longer in starvation conditions relative to CDX2(−) cells. Mouse experiments were then conducted in which CDX2 expression was introduced into CDX2(−) mice, that produced larger tumors that spread more aggressively, shortening survival (FIG. 3).

A database of drug-induced transcriptional effects was then analyzed to identify drugs that had the effect of inhibiting autophagy.

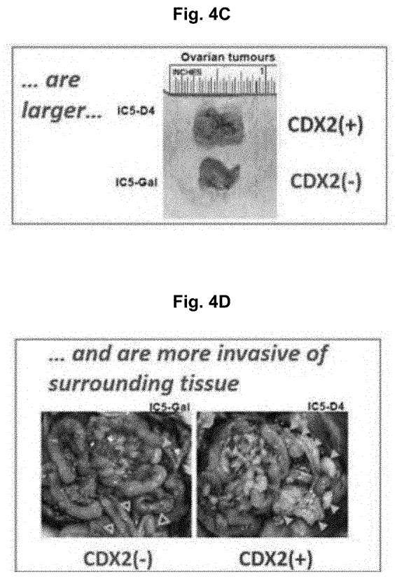

Three matches were found—the antimalarial agents chloroquine and mefloquine and the antihelminth lucanthone. Chloroquine was selected for experimental testing, and it was found that chloroquine did inhibit autophagy and reduce cancer cell survival in the hanging drop assay. Chloroquine treatment also improved survival in CDX2(+) mice, supporting the use of autophagy inhibitors in patients with CDX2(+) ovarian cancer (FIG. 6).

Autophagy inhibitors have been studied in cancer; however, their broad clinical use is restrained by an elevated risk of acute kidney injury. Autophagy has a renal protective role; hence, there is sensitivity to broadly inhibiting autophagy in patients while administering chemotherapy. In this sense, autophagy inhibition in cancer has been termed a “double-edged sword”. In high-grade serous ovarian cancer, CDX2 is overexpressed only in ˜10% of patients; hence the CDX2(+) minority would have a more favorable reward vs. risk balance than the ˜90% of patients who are CDX2(−). CDX2 is an essential biomarker for the appropriate use of autophagy inhibition to treat women with high-grade serous ovarian cancer.

### Example 2

Despite a solid knowledge of CDX2 binding partners and its targets in colon, AML and placental stem cells, the molecular pathways engaged by CDX2 in ovarian cancer remain unknown. The analysis identified significantly lower survival for patients with high CDX2 (FIG. 1A), but high CDX2 with wildtype (WT) TP53 significantly improved survival (FIG. 1C). High CDX2 denotes the expression level in for example the top 25 to 50% of the patients with ovarian cancer. Optionally the top 50%, or the top 25%.

The success of Herceptin for Her2-positive breast cancer patients⁴⁷⁻⁴⁹ highlights the benefit from identifying molecular markers to aid in diagnosis, prognosis and prediction of successful therapy response. As demonstrated herein, CDX2 over-expression identifies serous EOC patients with poor survival, and targeted treatment with chloroquine shows promising results (FIG. 1, 6). Importantly, CDX2 over-expression in endometrioid and mucinous EOC shows protective role, and P53 wild type renders high CDX2 in serous EOC protective.

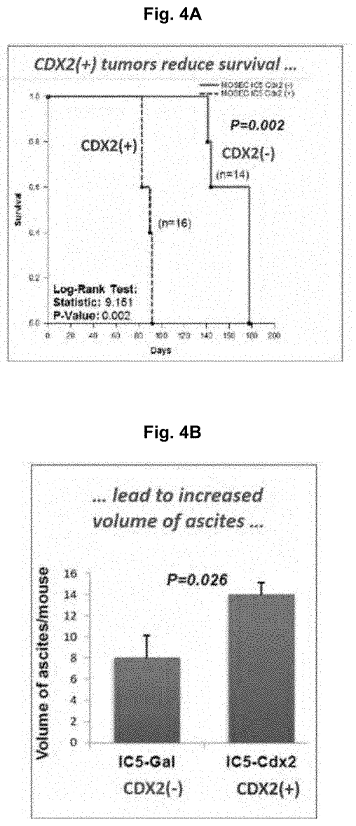

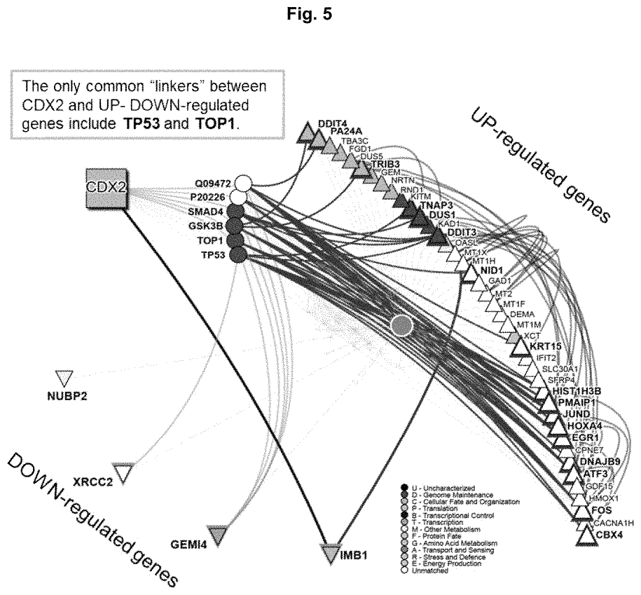

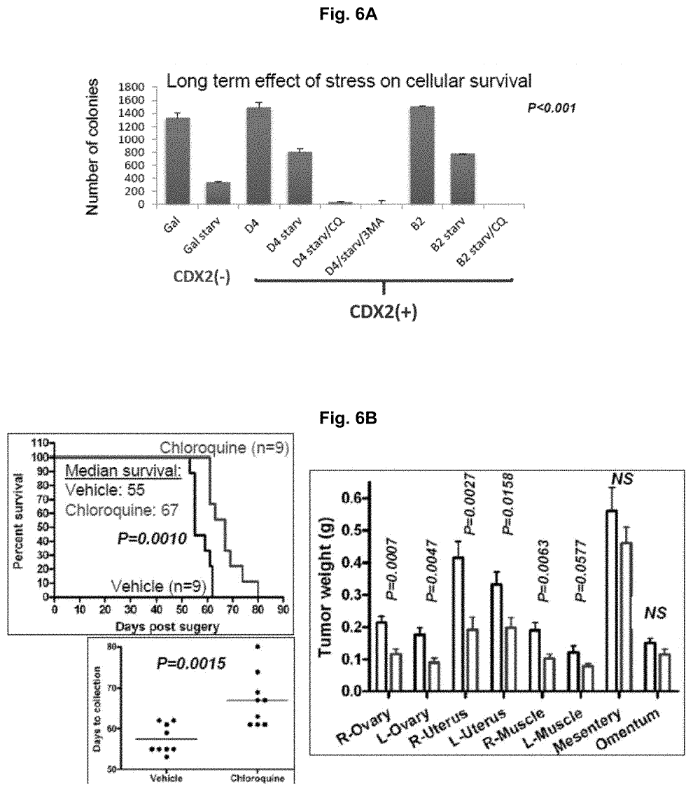

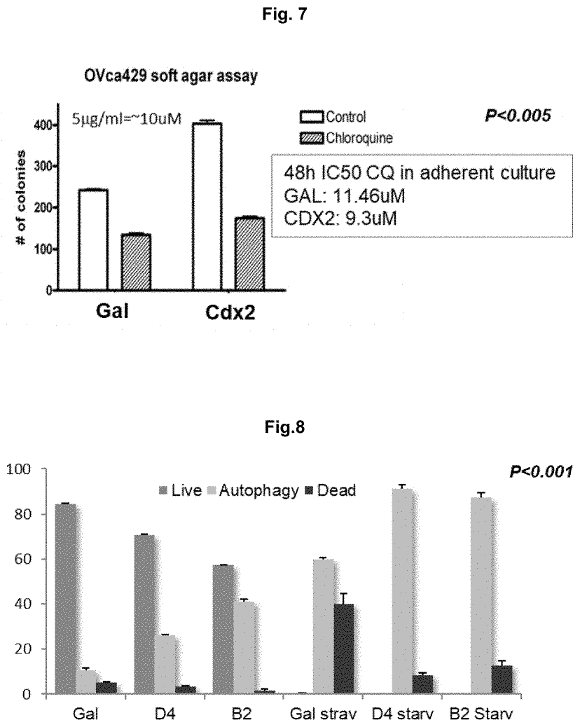

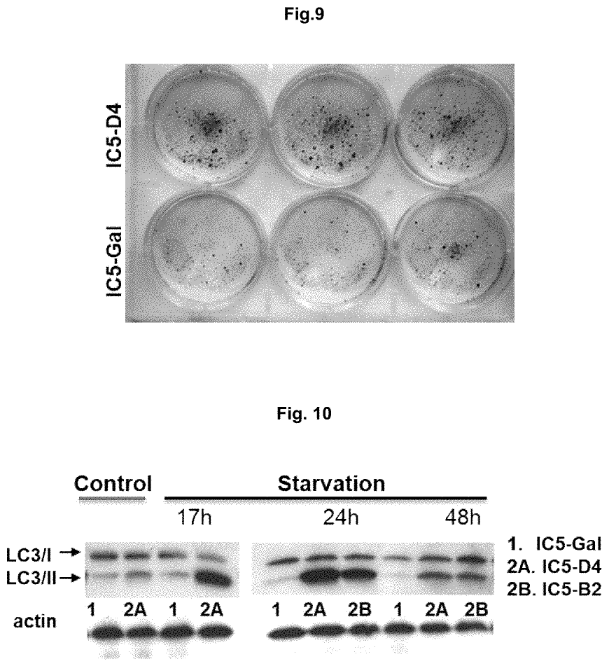

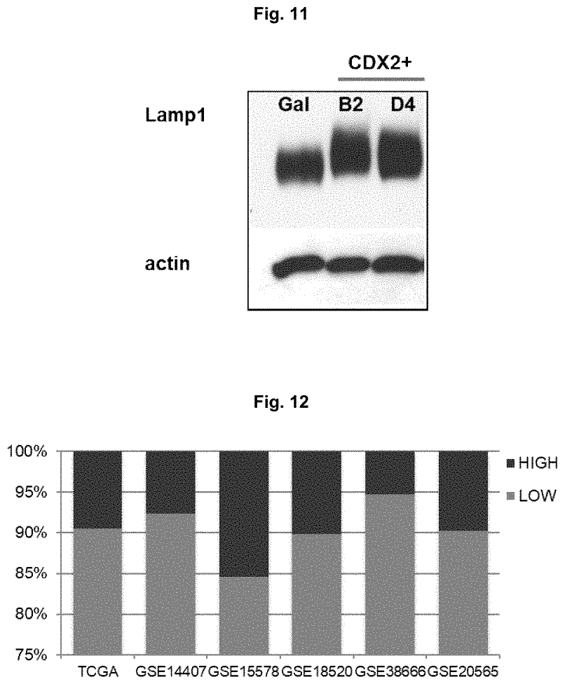

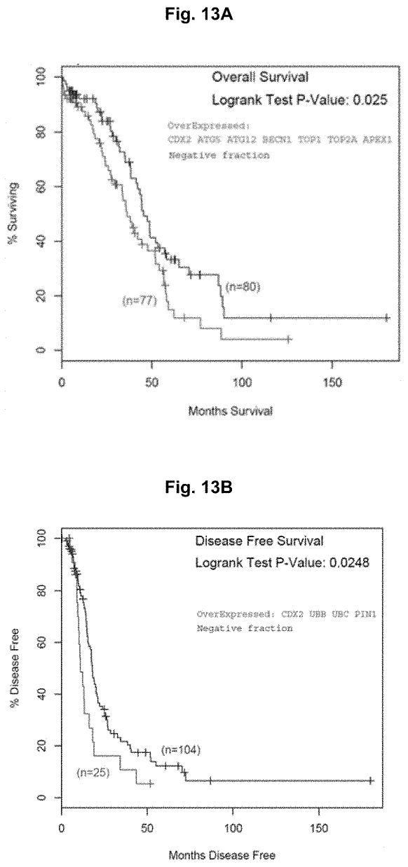

CDX2-expressing cells (serous human and murine) have elevated baseline levels of autophagy in vitro and in vivo, and increased colony formation assays after glucose and serum deprivation (FIG. 8-10). This is accompanied by elevated expression of autophagy markers (lipidation of LC31/II, decrease in p62). Chloroquine is a drug that affects lysosomal acidification, and thus interferes with autophagy. Applying a low dose of chloroquine abrogates cell survival (see IC50 FIG. 7). CDX2-expressing MOSEC cells have higher lysosomal content, contain hyperglycosylated lysosomal LAM P1 protein (FIG. 11), and their expression profile is enriched in lysosomal pathways (FIG. 5). Chloroquine has been determined to affect both parental line and CDX2-expressing cells with different IC50 doses (FIG. 7), indicating that EOC cells use autophagy for survival. Chloroquine decreased advantage of CDX2-driven anchorage-independent growth (4 weeks) in OVCA429 (FIG. 7).

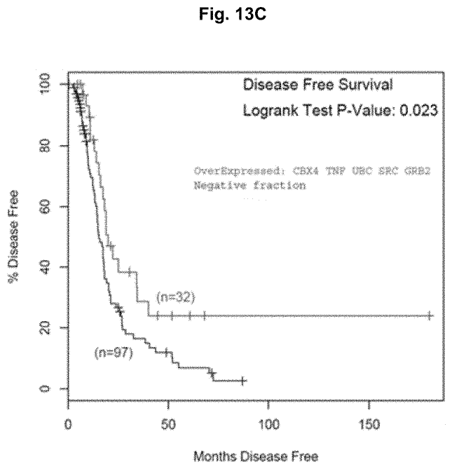

CDX2(+) show increased survival after long-term stress, but choloroquine (CQ) and the autophagy inhibitor 3-methyladenine (3MA) reverse this survival (FIG. 6A). It has been established that chloroquine alone (10 mg/kg 3 times a week) prolongs survival of mice with ovarian tumours (FIG. 6B) when administered when tumor is already established. Combination treatment may significantly enhance response, as platinum-based therapies have been shown to elicit autophagic response, which could be one of the mechanisms how EOC develops drug resistance. In addition, three autophagy genes are significantly up-regulated by chloroquine in Cdx2-expressing cells: NPC (adjusted P<0.02), SQSTM1 (adjusted P<0.02), and WIPI1 (adjusted P<0.03); while LAM P1 and ULK2 are down-regulated. Over-expression of additional genes identified from our networks significantly reduce patient survival (TOGA dataset [54]): over-expressed Cdx2 autophagy genes (Cdx2, Atg5, Atg12, Becn1) and lysosomal/autophagy genes (Top1, Top2A, Apex1) P<0.025 and saw significantly lower overall survival(FIG. 13A) ; over-expressed lysosome related genes and Cdx2(Cdx2, Ubb, Ubc, Pin1) P<0.0248 and showed significantly lower disease-free survival (FIG. 13B). Importantly, genes over-expressed by chloroquine treatment (Cbx4, Tnf, Ubc, Src, Grb2) are linked to significantly improved disease-free survival (TOGA dataset [54]; P<0.023; FIG. 13C).

All selected compounds trigger significantly overlapping changes in gene expression patterns (in addition to drug-specific transcriptional responses), and thus we will explore them individually and in combination with standard therapy for efficacy of CDX2-expressing EOC growth. Without wishing to be bound by theory, several analyses determined that chloroquine regulates fewer genes than mefloquine or halofantrine, and thus may induce fewer side-effects. We will determine whether CDX2-expressing ovarian cancer cells better survive nutritional deprivation due to activation of autophagy. We will assess whether interference with lysosomal function sensitizes them for preferential elimination, and whether this proves beneficial as synergistic treatment option in combination therapy.

It was determined that chloroquine regulates fewer genes than mefloquine or halofantrine, and thus will likely induce fewer side-effects.

### Significance of CDX2 as a Prognostic/Predictive Marker in EOC.

CDX2 over-expression in diverse epithelial cancers had been established, yet its clinical significance as a prognostic marker, and its role in tumour behavior remains unclear (Table 1). Its over-expression in colon and pancreatic ductal carcinomas predicts improved patient survival, but CDX2 up-regulation in AML is not favourable³⁷. Computational analysis of publicly available dataset as well as histological TMA scoring confirmed significantly better prognosis for patients with tumors expressing CDX2 in endometrioid subtype. Re-analysis of HGSC publicly available datasets revealed CDX2 over-expression in ˜10% of patients with high grade serous epithelial ovarian cancer (FIG. 13), and overall these have significantly shorter disease free survival period (FIG. 1A). However, when status of p53 (mutated or WT) was taken into account, interesting pattern emerged. While patients with WT p53 status and over-expression of CDX2 had better prognosis (HR=0.59; P=0.078;1C), p53 mutated background had significantly shorter disease-free survival (HR=1.64; P<0.042;FIG. 1B). This indicates biological interaction between CDX2 and p53.

TABLE 1
CDX2 expression, its impact on survival, and
its role and mechanism differ across cancers

| Neoplasm | mRNA | Survival | Role | Mechanism |
|---|---|---|---|---|
| Acute myeloid | HIGH | POOR | Tumor | Differentiation, |
| leukemia | expression | Survival | initiating | proliferation |
| Colon cancer | HIGH | Unknown | Promotes | Anchorage- |
| cell lines | expression | | growth | independent |
| | | | | growth |
| Colorectal | LOW | POOR | Tumor | Down/poor |
| cancer | expression | survival | suppressor | differentiation |
| Esophageal | Progressive | Unknown | Promotes | Promotes |
| cancer | gain | | growth | metaplasia |
| Gallbladder | Variable | Unknown | Unknown | Inconsistent |
| adenocarci- | | | | |
| noma | | | | |
| Gastric cancer | LOW | POOR | Suppresses | Differentiation |
| | expression | survival | metastasis | |
| Pancreatic | LOW | POOR | Unknown | Unknown |
| cancer | expression | survival | | |

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### CITATIONS FOR REFERENCES REFERRED TO IN THE SPECIFICATION

1. Statistics, C.C.S.s.S.C.o.C., Edn. May 2012 (Canadian Cancer Society, Statistics Canada, Toronto, ON; 2012).
2. Fields, M. M. & Chevlen, E. Screening for disease: making evidence-based choices. Clin J Oncol Nurs 10, 73-76 (2006).
3. Fields, M. M. & Chevlen, E. Ovarian cancer screening: a look at the evidence. Clin J Oncol Nurs 10, 77-81 (2006).
4. Shih le, M. & Kurman, R. J. Ovarian tumorigenesis: a proposed model based on morphological and molecular genetic analysis. Am J Pathol 164, 1511-1518 (2004).
5. Shih le, M. & Kurman, R. J. Molecular pathogenesis of ovarian borderline tumors: new insights and old challenges. Clin Cancer Res 11, 7273-7279 (2005).
6. Wojnarowicz, P. M. et al. The genomic landscape of TP53 and p53 annotated high grade ovarian serous carcinomas from a defined founder population associated with patient outcome. PloS one 7, e45484 (2012).
7. Patrono, M. G. et al. Borderline tumours of the ovary, current controversies regarding their diagnosis and treatment. Ecancermedicalscience 7, 379 (2013).
8. Lalwani, N. et al. Current update on borderline ovarian neoplasms. AJR. American journal of roentgenology 194, 330-336 (2010).
9. Vaughan, S. et al. Rethinking ovarian cancer: recommendations for improving outcomes. Nature reviews. Cancer 11, 719-725 (2011).
10. Auersperg, N., Ota, T. & Mitchell, G. W. Early events in ovarian epithelial carcinogenesis: progress and problems in experimental approaches. Int J Gynecol Cancer 12, 691-703 (2002).
11. Colgan, T. J. Challenges in the early diagnosis and staging of Fallopian-tube carcinomas associated with BRCA mutations. Int J Gynecol Pathol 22, 109-120 (2003).
12. Folkins, A. K. et al. A candidate precursor to pelvic serous cancer (p53 signature) and its prevalence in ovaries and fallopian tubes from women with BRCA mutations. Gynecol Oncol 109, 168-173 (2008).
13. Lee, Y. et al. Advances in the recognition of tubal intraepithelial carcinoma: applications to cancer screening and the pathogenesis of ovarian cancer. Adv Anat Pathol 13, 1-7 (2006).
14. Lee, Y. et al. A candidate precursor to serous carcinoma that originates in the distal fallopian tube. J Pathol 211, 26-35 (2007).
15. Cheng, W., Liu, J., Yoshida, H., Rosen, D. & Naora, H. Lineage infidelity of epithelial ovarian cancers is controlled by HOX genes that specify regional identity in the reproductive tract. Nature medicine 11, 531-537 (2005).
16. Strumpf, D. et al. Cdx2 is required for correct cell fate specification and differentiation of trophectoderm in the mouse blastocyst. Development 132, 2093-2102 (2005).
17. Gao, N. & Kaestner, K. H. Cdx2 regulates endo-lysosomal function and epithelial cell polarity. Genes & development 24, 1295-1305 (2010).
18. Hryniuk, A., Grainger, S., Savory, J. G. & Lohnes, D. Cdx function is required for maintenance of intestinal identity in the adult. Developmental biology 363, 426-437 (2012).
19. Renouf, B. et al. Cdx2 homeoprotein inhibits non-homologous end joining in colon cancer but not in leukemia cells. Nucleic Acids Res 40, 3456-3469 (2012).
20. Verzi, M. P. et al. Differentiation-specific histone modifications reveal dynamic chromatin interactions and partners for the intestinal transcription factor CDX2. Developmental cell 19, 713-726 (2010).
21. Verzi, M. P., Shin, H., San Roman, A. K., Liu, X. S. & Shivdasani, R. A. Intestinal master transcription factor CDX2 controls chromatin access for partner transcription factor binding. Molecular and Cellular Biology 33, 281-292 (2013).
22. Chawengsaksophak, K., James, R., Hammond, V. E., Kontgen, F. & Beck, F. Homeosis and intestinal tumours in Cdx2 mutant mice. Nature 386, 84-87 (1997).
23. Bonhomme, C. et al. The Cdx2 homeobox gene has a tumour suppressor function in the distal colon in addition to a homeotic role during gut development. Gut 52, 1465-1471 (2003).
24. Gangoda, L. et al. Cre transgene results in global attenuation of the cAMP/PKA pathway. Cell Death Dis 3, e365 (2012).
25. Dang, L. H. et al. CDX2 has tumorigenic potential in the human colon cancer cell lines LOVO and SW48. Oncogene 25, 2264-2272 (2006).
26. Barros, R., Freund, J. N., David, L. & Almeida, R. Gastric intestinal metaplasia revisited: function and regulation of CDX2. Trends in molecular medicine 18, 555-563 (2012).
27. Moskaluk, C. A. et al. Cdx2 protein expression in normal and malignant human tissues: an immunohistochemical survey using tissue microarrays. Modern pathology: an official journal of the United States and Canadian Academy of Pathology, Inc 16, 913-919 (2003).
28. Baker, M. L. et al. Invasive mucinous cystic neoplasms of the pancreas. Experimental and molecular pathology (2012).
29. Yeh, T. S. et al. Aberrant expression of cdx2 homeobox gene in intraductal papillary-mucinous neoplasm of the pancreas but not in pancreatic ductal adenocarcinoma. Pancreas 30, 233-238 (2005).
30. Park, K. J., Bramlage, M. P., Ellenson, L. H. & Pirog, E. C. Immunoprofile of adenocarcinomas of the endometrium, endocervix, and ovary with mucinous differentiation. Appl lmmunohistochem Mol Morphol 17, 8-11 (2009).
31. Sullivan, L. M., Smolkin, M. E., Frierson, H. F., Jr. & Galgano, M. T. Comprehensive evaluation of CDX2 in invasive cervical adenocarcinomas: immunopositivity in the absence of overt colorectal morphology. The American journal of surgical pathology 32, 1608-1612 (2008).
32. Borrisholt, M., Nielsen, S. & Vyberg, M. Demonstration of CDX2 is Highly Antibody Dependant. Appl Immunohistochem Mol Morphol (2012).
33. Li, Q. L., Yang, Z. L., Liu, J. Q. & Miao, X. Y. Expression of CDX2 and hepatocyte antigen in benign and malignant lesions of gallbladder and its correlation with histopathologic type and clinical outcome. Pathology oncology research: POR 17, 561-568 (2011).
34. Riedt, T. et al. Aberrant expression of the homeobox gene CDX2 in pediatric acute lymphoblastic leukemia. Blood 113, 4049-4051 (2009).
35. Scholl, C. et al. The homeobox gene CDX2 is aberrantly expressed in most cases of acute myeloid leukemia and promotes leukemogenesis. The Journal of clinical investigation 117, 1037-1048 (2007).
36. Baba, Y. et al. Relationship of CDX2 loss with molecular features and prognosis in colorectal cancer. Clin Cancer Res 15, 4665-4673 (2009).
37. Thoene, S. et al. The homeobox gene CDX2 is aberrantly expressed and associated with an inferior prognosis in patients with acute lymphoblastic leukemia. Leukemia: official journal of the Leukemia Society of America, Leukemia Research Fund, U.K 23, 649-655 (2009).
38. Babelova, A., Sedding, D. G. & Brandes, R. P. Anti-atherosclerotic mechanisms of statin therapy. Current opinion in pharmacology (2013).
39. Faber, K. et al. CDX2-driven leukemogenesis involves KLF4 repression and deregulated PPARgamma signaling. The Journal of clinical investigation 123, 299-314 (2013).
40. Nishiyama, A. et al. Uncovering early response of gene regulatory networks in ESCs by systematic induction of transcription factors. Cell stem cell 5, 420-433 (2009).
41. Fraggetta, F. et al. CDX2 immunoreactivity in primary and metastatic ovarian mucinous tumours. Virchows Arch 443, 782-786 (2003).
42. Vang, R. et al. Immunohistochemical expression of CDX2 in primary ovarian mucinous tumors and metastatic mucinous carcinomas involving the ovary: comparison with CK20 and correlation with coordinate expression of CK7. Modern pathology: an official journal of the United States and Canadian Academy of Pathology, Inc 19, 1421-1428 (2006).
43. Feng, H., Ghazizadeh, M., Konishi, H. & Araki, T. Expression of MUC1 and MUC2 mucin gene products in human ovarian carcinomas. Japanese journal of clinical oncology 32, 525-529 (2002).
44. Yoon, O. & Roh, J. Downregulation of KLF4 and the Bcl-2/Bax ratio in advanced epithelial ovarian cancer. Oncol Lett 4, 1033-1036 (2012).
45. Kleinberg, L., Holth, A., Trope, C. G., Reich, R. & Davidson, B. Claudin upregulation in ovarian carcinoma effusions is associated with poor survival. Human pathology 39, 747-757 (2008).
46. Konecny, G. E. et al. Claudin-3 and claudin-4 expression in serous papillary, clear-cell, and endometrioid endometrial cancer. Gynecologic oncology 109, 263-269 (2008).
47. Pegram, M. D. et al. Phase II study of receptor-enhanced chemosensitivity using recombinant humanized anti-p185HER2/neu monoclonal antibody plus cisplatin in patients with HER2/neu-overexpressing metastatic breast cancer refractory to chemotherapy treatment. J Clin Oncol 16, 2659-2671 (1998).
48. Slamon, D. Herceptin: increasing survival in metastatic breast cancer. Eur J Oncol Nurs 4, 24-29 (2000).
49. Vogel, C. L. et al. First-line Herceptin monotherapy in metastatic breast cancer. Oncology 61 Suppl 2, 37-42 (2001).
50. Roby, K. F. et al. Development of a syngeneic mouse model for events related to ovarian cancer. Carcinogenesis 21, 585-591 (2000).
51. Greenaway, J., Moorehead, R., Shaw, P. & Petrik, J. Epithelial-stromal interaction increases cell proliferation, survival and tumorigenicity in a mouse model of human epithelial ovarian cancer. Gynecol Oncol 108, 385-394 (2008).
52. Urzua, U. et al. Microarray comparative genomic hybridization profile of a murine model for epithelial ovarian cancer reveals genomic imbalances resembling human ovarian carcinomas. Tumour Biol 26, 236-244 (2005).
53. DiCioccio, R. A., Werness, B. A., Peng, R., Allen, H. J. & Piver, M. S. Correlation of TP53 mutations and p53 expression in ovarian tumors. Cancer genetics and cytogenetics 105, 93-102 (1998).
54. TCGA_consortium Integrated genomic analyses of ovarian carcinoma. Nature 474, 609-615 (2011).
55. Tothill, R. W. et al. Novel molecular subtypes of serous and endometrioid ovarian cancer linked to clinical outcome. Clin Cancer Res 14, 5198-5208 (2008).
56. Fortney, K., Kotlyar, M. & Jurisica, I. Inferring the functions of longevity genes with modular subnetwork biomarkers of Caenorhabditis elegans aging. Genome Biol 11, R13 (2010).
57. Fortney, K., Morgen, E. K., Kotlyar, M. & Jurisica, I. In silico drug screen in mouse liver identifies candidate calorie restriction mimetics. Rejuvenation research 15, 148-152 (2012).
58. Kotlyar, M., Fortney, K. & Jurisica, I. Network-based characterization of drug-regulated genes, drug targets, and toxicity. Methods (2012).
59. Fortney, K. et al. NetwoRx: connecting drugs to networks and phenotypes in Saccharomyces cerevisiae. Nucleic Acids Res 41, D720-727 (2013).
60. Pastrello, C. et al. Visual data mining of biological networks: one size does not fit all. PLoS computational biology 9, e1002833 (2013).
61. Lapin, V. et al. Kinome-wide screening of HER2+ breast cancer cells for molecules that mediate cell proliferation or sensitize cells to trastuzumab therapy. Oncogenesis 3, e133 (2014).
62. Petschnigg, J. et al. The mammalian-membrane two-hybrid assay (MaMTH) for probing membrane-protein interactions in human cells. Nat Methods 11, 585-592 (2014).
63. Rawat, V. P. et al. Ectopic expression of the homeobox gene Cdx2 is the transforming event in a mouse model of t(12;13)(p13;q12) acute myeloid leukemia. Proceedings of the National Academy of Sciences of the United States of America 101, 817-822 (2004).
64. Flesken-Nikitin, A. et al. Ovarian surface epithelium at the junction area contains a cancer-prone stem cell niche. Nature 495, 241-245 (2013).
65. Barker, N. et al. Identification of stem cells in small intestine and colon by marker gene Lgr5. Nature 449, 1003-1007 (2007).
66. Bouchard, M., Souabni, A. & Busslinger, M. Tissue-specific expression of cre recombinase from the Pax8 locus. Genesis 38, 105-109 (2004).
67. Jamin, S. P., Arango, N. A., Mishina, Y., Hanks, M. C. & Behringer, R. R. Requirement of Bmpr1a for Mullerian duct regression during male sexual development. Nature Genetics 32, 408-410 (2002).
68. Kim, J. et al. High-grade serous ovarian cancer arises from fallopian tube in a mouse model. Proceedings of the National Academy of Sciences of the United States of America 109, 3921-3926 (2012).
69. Mullany, L. K. et al. Molecular and functional characteristics of ovarian surface epithelial cells transformed by KrasG12D and loss of Pten in a mouse model in vivo. Oncogene 30, 3522-3536 (2011).
70. Perets, R. et al. Transformation of the fallopian tube secretory epithelium leads to high-grade serous ovarian cancer in Brca;Tp53;Pten models. Cancer Cell 24, 751-765 (2013).
71. Rawat, V. P. et al. Overexpression of CDX2 perturbs HOX gene expression in murine progenitors depending on its N-terminal domain and is closely correlated with deregulated HOX gene expression in human acute myeloid leukemia. Blood 111, 309-319 (2008).
72. Vucic, E. A. et al. Smoking status impacts microRNA mediated prognosis and lung adenocarcinoma biology. BMC Cancer 14, 778 (2014).
73. Der, S. D. et al. Validation of a histology-independent prognostic gene signature for early-stage, non-small-cell lung cancer including stage IA patients. Journal of thoracic oncology: official publication of the International Association for the Study of Lung Cancer 9, 59-64 (2014).
74. Cervigne, N. K. et al. Recurrent genomic alterations in sequential progressive leukoplakia and oral cancer: drivers of oral tumorigenesis? Human molecular genetics 23, 2618-2628 (2014).
75. Goswami, R. S. et al. MicroRNA Signature Obtained From the Comparison of Aggressive With Indolent Non-Hodgkin Lymphomas: Potential Prognostic Value in Mantle-Cell Lymphoma. Journal of clinical oncology: official journal of the American Society of Clinical Oncology 31, 2903-2911 (2013).
76. Reis, P. P. et al. A gene signature in histologically normal surgical margins is predictive of oral carcinoma recurrence. BMC Cancer 11, 437 (2011).
77. Agarwal, R., Jurisica, I., Mills, G. B. & Cheng, K. W. The emerging role of the RAB25 small GTPase in cancer. Traffic 10, 1561-1568 (2009).
78. Zhu, C. Q. et al. Understanding prognostic gene expression signatures in lung cancer. Clin Lung Cancer 10, 331-340 (2009).
79. Boutros, P. C. et al. Re: Gene expression-based prognostic signatures in lung cancer: ready for clinical use? J Natl Cancer Inst 102, 1677-1678; author reply 1678-1679 (2010).
80. Goswami, R. S. et al. Optimization and analysis of a quantitative real-time PCR-based technique to determine microRNA expression in formalin-fixed paraffin-embedded samples. BMC Biotechnol 10, 47 (2010).
81. Hui, A. B. et al. Comprehensive MicroRNA Profiling for Head and Neck Squamous Cell Carcinomas. Clin Cancer Res 16, 1129-1139 (2010).
82. Shedden, K. et al. Gene expression-based survival prediction in lung adenocarcinoma: a multi-site, blinded validation study. Nat Med 14, 822-827 (2008).
83. Navab, R. et al. Prognostic gene-expression signature of carcinoma-associated fibroblasts in non-small cell lung cancer. Proc Natl Acad Sci USA 108, 7160-7165 (2011).
84. Eppert, K. et al. Stem cell gene expression programs influence clinical outcome in human leukemia. Nat Med 17, 1086-1093 (2011).
85. Elschenbroich, S. et al. In-depth proteomics of ovarian cancer ascites: combining shotgun proteomics and selected reaction monitoring mass spectrometry. J Proteome Res 10, 2286-2299 (2011).
86. Chang, Q., Jurisica, I., Do, T. & Hedley, D. W. Hypoxia predicts aggressive growth and spontaneous metastasis formation from orthotopically grown primary xenografts of human pancreatic cancer. Cancer Res 71, 3110-3120 (2011).
87. Fortney, K. & Jurisica, I. Integrative computational biology for cancer research. Hum Genet (2011).
88. Zhu, C. Q. et al. Prognostic and predictive gene signature for adjuvant chemotherapy in resected non-small-cell lung cancer. J Clin Oncol 28, 4417-4424 (2010).
89. Zhu, C. Q. et al. Prognostic Gene Expression Signature for Squamous Cell Carcinoma of Lung. Clin Cancer Res (2010).
90. Reis, P. P. et al. Programmed cell death 4 loss increases tumor cell invasion and is regulated by miR-21 in oral squamous cell carcinoma. Mol Cancer 9, 238 (2010).
91. Radulovich, N. et al. Differential roles of cyclin D1 and D3 in pancreatic ductal adenocarcinoma. Mol Cancer 9, 24 (2010).
92. Savas, S., Geraci, J., Jurisica, I. & Liu, G. A comprehensive catalogue of functional genetic variations in the EGFR pathway: protein-protein interaction analysis reveals novel genes and polymorphisms important for cancer research. Int J Cancer 125, 1257-1265 (2009).
93. Mills, G. B., Jurisica, I., Yarden, Y. & Norman, J. C. Genomic amplicons target vesicle recycling in breast cancer. J Clin Invest 119, 2123-2127 (2009).
94. Boutros, P. C. et al. Prognostic gene signatures for non-small-cell lung cancer. Proc Natl Acad Sci USA 106, 2824-2828 (2009).
95. Sodek, K. L. et al. Identification of pathways associated with invasive behavior by ovarian cancer cells using multidimensional protein identification technology (MudPIT). Mol Biosyst 4, 762-773 (2008).
96. Gortzak-Uzan, L. et al. A proteome resource of ovarian cancer ascites: integrated proteomic and bioinformatic analyses to identify putative biomarkers. J Proteome Res 7, 339-351 (2008).
97. Orchard, S. et al. Protein interaction data curation: the International Molecular Exchange (IMEx) consortium. Nat Methods 9, 345-350 (2012).
98. Brown, K. R. & Jurisica, I. Online predicted human interaction database. Bioinformatics 21, 2076-2082 (2005).
99. Brown, K. R. & Jurisica, I. Unequal evolutionary conservation of human protein interactions in interologous networks. Genome Biol (2007).
100. Niu, Y., Otasek, D. & Jurisica, I. Evaluation of linguistic features useful in extraction of interactions from PubMed; application to annotating known, high-throughput and predicted interactions in I2D. Bioinformatics 26, 111-119 (2010).
101. Kotlyar, M. & Jurisica, I. Predicting protein-protein interactions by association mining. Information Systems Frontiers 8, 37-47 (2006).
102. Kotlyar, M. et al. In silico prediction of physical protein interactions and characterization of interactome orphans. Nat Methods 12, 79-84 (2015).
103. Vafaee, F., Rosu, D., Broackes-Carter, F. & Jurisica, I. Novel semantic similarity measure improves an integrative approach to predicting gene functional associations. BMC Syst Biol (2013).
104. Dingar, D. et al. BioID identifies novel c-MYC interacting partners in cultured cells and xenograft tumors. J Proteomics (2014).
105. Tu, W. B. et al. Myc and its interactors take shape. Biochimica et biophysica acta (2014).
106. Deribe, Y. L. et al. Regulation of epidermal growth factor receptor trafficking by lysine deacetylase HDAC6. Science signaling 2, ra84 (2009).
107. Kittanakom, S. et al. CHIP-MYTH: a novel interactive proteomics method for the assessment of agonist-dependent interactions of the human beta(2)-adrenergic receptor. Biochemical and biophysical research communications 445, 746-756 (2014).
108. Yang, S. et al. Pancreatic cancers require autophagy for tumor growth. Genes & development 25, 717-729 (2011).
112. Johansen, T. & Lamark, T. Selective autophagy mediated by autophagic adapter proteins. Autophagy 7, 279-296 (2011).
113. Pankiv, S. et al. p62/SQSTM1 binds directly to Atg8/LC3 to facilitate degradation of ubiquitinated protein aggregates by autophagy. The Journal of biological chemistry 282, 24131-24145 (2007).
114. Fraldi, A. et al. Lysosomal fusion and SNARE function are impaired by cholesterol accumulation in lysosomal storage disorders. The EMBO journal 29, 3607-3620 (2010).
115. Sukhai, M. A. et al. Lysosomal disruption preferentially targets acute myeloid leukemia cells and progenitors. The Journal of clinical investigation 123, 315-328 (2013).
116. Lamb, J. et al. The Connectivity Map: using gene-expression signatures to connect small molecules, genes, and disease. Science 313, 1929-1935 (2006).
117. Wishart, D. S. et al. DrugBank: a comprehensive resource for in silico drug discovery and exploration. Nucleic Acids Res 34, D668-672 (2006).
118. Wishart, D. S. DrugBank and its relevance to pharmacogenomics. Pharmacogenomics 9, 1155-1162 (2008).
119. Heifets, A. & Jurisica, I. SCRIPDB: a portal for easy access to syntheses, chemicals and reactions in patents. Nucleic Acids Res (2011).
120. Rabinowitz, J. D. & White, E. Autophagy and metabolism. Science 330, 1344-1348 (2010). 121. Del Rowe, J. D. et al. Accelerated regression of brain metastases in patients receiving whole brain radiation and the topoisomerase II inhibitor, lucanthone. International journal of radiation oncology, biology, physics 43, 89-93 (1999).
122. Xu, Y. et al. Inhibition of autophagy enhances cisplatin cytotoxicity through endoplasmic reticulum stress in human cervical cancer cells. Cancer letters 314, 232-243 (2012).
123. Rosenfeldt, M. T. et al. p53 status determines the role of autophagy in pancreatic tumour development. Nature 504, 296-300 (2013).

1. A method of selecting a patient afflicted with high-grade serous epithelial ovarian cancer likely to benefit from an autophagy inhibitor treatment, the method comprising:
a. obtaining a biological sample;
b. testing the biological sample for upregulated CDX2; and
c. selecting the patient having upregulated CDX2 as likely to benefit from and/or for treatment with the autophagy inhibitor treatment.

2. The method of claim 1 further comprising testing for loss of function of p53 protein, optionally testing for one or more deleterious mutations in p53, and selecting the patient having upregulated CDX2 and loss of function of the p53 protein for treatment with the autophagy inhibitor treatment.

3. The method of claim 1, wherein the autophagy inhibitor treatment targets PI3 kinase, autophagosome-lysosome fusion and/or autolysosomal function.

4. The method of claim 1, wherein the autophagy inhibitor treatment comprises an autophagy inhibitor selected from ROC-325, 3-methyladenine, chloroquine, mefloquine, or lucanthone, preferably 3-methyladenine and/or chloroquine, or their analogs and/or combinations thereof.

5. The method of claim 1, wherein the biological sample is a tumor sample, optionally a surgical sample, an ascites cellular fraction, or a liquid biopsy comprising circulating tumor cells or circulating tumor DNA.

6. The method of claim 1, wherein the biological sample comprises cancer cell nucleic acids and/or a protein fraction.

7. The method of claim 1, wherein the testing comprises measuring the cellular levels of the CDX2 protein and/or mRNA, optionally wherein the cellular levels of the CDX2 mRNA are detected by RT-PCR or qPCR methods and/or the cellular levels of the of the CDX2 protein are detected using a standard polypeptide assay, optionally an ELISA or western blot, or by immunohistochemistry of a tumor sample or immunocytochemistry of a cell sample.

8. The method of claim 1, wherein the testing comprises assessing for amplification of CDX2, optionally using qPCR, RNAseq, and/or FISH.

9. The method of claim 1, wherein the testing comprises assaying for one or more deleterious mutations in p53, optionally sequencing a p53 transcript or part thereof and optionally comparing the p53 transcript sequence to wild type p53 to identify the presence or absence of one or more deleterious mutations.

10. A method of treating a patient afflicted with high-grade serous epithelial ovarian cancer having upregulated CDX2, the method comprising administering to the patient an autophagy inhibitor treatment.

11. The method of claim 10, wherein the patient has a loss of function of p53 protein and/or one or more deleterious mutations in p53.

12. The method of claim 10, wherein the autophagy inhibitor treatment targets PI3 kinase, autophagosome-lysosome fusion and/or autolysosomal function.

13. The method of claim 10, wherein the autophagy inhibitor treatment comprises an autophagy inhibitor selected from ROC-325, chloroquine, mefloquine, lucanthone, preferably 3-methyladenine and/or chloroquine, or their analogs or combinations thereof.

14. A method of treating a patient afflicted with high-grade serous epithelial ovarian cancer, the method comprising:
a. obtaining a biological sample;
b. testing the biological sample for upregulated CDX2; and
c. treating the patient having upregulated CDX2, with an autophagy inhibitor treatment.

15. The method of claim 14 further comprising testing for loss of function of p53 protein, optionally testing for one or more deleterious mutations in p53 and treating the patient having upregulated CDX2 and loss of function of the p53 protein.

16. The method of claim 14, wherein the autophagy inhibitor treatment targets PI3 kinase, autophagosome-lysosome fusion and/or autolysosomal function.

17. The method of claim 14, wherein the autophagy inhibitor treatment comprises an autophagy inhibitor selected from ROC-325, chloroquine, mefloquine, or lucanthone, preferably 3-methyladenine and/or chloroquine, or their analogs or combinations thereof.

18. The method of claim 14, wherein the biological sample is a tumor sample, optionally a surgical sample, an ascites cellular fraction, or a liquid biopsy comprising circulating tumor cells or circulating tumor DNA.

19. The method of claim 14, wherein the biological sample comprises cancer cell nucleic acids and/or a protein fraction.

20. The method of claim 14, wherein the testing comprises:
i) measuring cellular levels of the CDX2 protein or mRNA, optionally wherein the cellular levels of the CDX2 mRNA are detected by RT-PCR or qPCR methods or wherein the cellular levels of the CDX2 protein are detected using a standard polypeptide assay, or by immunohistochemistry of a tumor sample or immunohistochemistry of a cell sample;
ii) assessing for amplification of CDX2 or a mutant CDX2, optionally wherein the amplification of CDX2 is detected using qPCR, RNAseq, and/or FISH; or
iii) assaying for one or more deleterious mutations in p53, optionally sequencing a p53 transcript or part thereof and optionally comparing the p53 transcript sequence to wild type p53 to identify the presence or absence of one or more deleterious mutations.
